(19) United States
(12) Patent Application Publication
(10) Pub. No.: US 2026/0315602 A1
(43) Pub. Date: Oct. 15, 2026

(54) USES OF AMINO ACID-CHELATED ZINC IN TREATING LUNG CANCER

(71) Applicant: FAAC Bio Inc., New Taipei City (TW)

(72) Inventors: HSIN-CHING HSU, New Taipei City (TW); LI-CHUAN HSU, New Taipei City (TW); WEI-YU LIN, New Taipei City (TW); HAO-CHUAN FU, New Taipei City (TW)

(21) Appl. No.: 19/255,444

(22) Filed: Jun. 30, 2025

(30) Foreign Application Priority Data
Apr. 9, 2025 (TW) 114113406

(51) Int. Cl. A61K 47/54

(52) U.S. Cl. CPC A61K 47/547

(57) ABSTRACT

A method for treating a lung cancer is provided. The method for treating the lung cancer that is provided is especially suited for treating a lung cancer with an EGFR (Epidermal Growth Factor Receptor) gene mutation or a lung cancer with a resistance to epidermal growth factor receptor tyrosine kinase inhibitors (EGFR-TKIs).

### CLAIM FOR PRIORITY

This application claims the benefits of Taiwan Patent Application No. 114113406 filed on Apr. 9, 2025, the subject matters of which are incorporated herein in their entirety by reference.

### FIELD OF THE INVENTION

The present invention relates to use of amino acid-chelated zinc, especially the use of amino acid-chelated zinc on treating a lung cancer in a subject. The present invention particularly relates to the use of amino acid-chelated zinc on treating a lung cancer or alleviating the condition of lung cancer in a subject, wherein the lung cancer has an Epidermal Growth Factor Receptor (EGFR) gene mutation or a drug resistance to epidermal growth factor receptor tyrosine kinase inhibitors (EGFR-TKIs).

### BACKGROUND OF THE INVENTION

Lung cancer is a malignant tumor located in lung and bronchus. Depending on the differences of biological properties and clinical performance, lung cancer can be classified into small cell lung cancer (SCLC) and non-small cell lung cancer (NSCLC), wherein the NSCLC further includes adenocarcinoma, squamous cell carcinoma and large cell carcinoma. The American Lung Association classifies the NSCLC into Stages 0 to IV based on the TNM (tumor-node-metastasis) system, wherein Stage 0 refers to an early-stage lung cancer confined to the top lining of the lung or bronchus without any spread, Stage I (including Stage IA and Stage IB) refers to a lung cancer that has not spread to lymph nodes or other parts of the body, Stage II (including Stage IIA and Stage IIB) refers to a larger tumor and/or a lung cancer that has spread to nearby lymph nodes but not to distant organs, Stage III (including Stage IIIA, Stage IIIB and Stage IIIC) refers to a lung cancer that has spread to the lymph nodes in the mediastinum (the area in the chest between the left and right lungs), and Stage IV refers to a terminal lung cancer that has metastasized or spread to the lining of the lung or other areas of the body.

The lung cancer stage is associated with patient survival rates. Taking the five-year survival rate as an example, Stage IA patients have a survival rate ranging from about 61% to 67%, Stage IB patients have a survival rate ranging from about 38% to 57%, Stage II patients have a survival rate ranging from about 24% to 55%, Stage IIIA patients have a survival rate only ranging from about 5% to 23%, and Stage IIIB and Stage IV patients have a survival rate even less than 5%. Since early-stage lung cancer often presents no significant symptoms, it is difficult to detect abnormalities in patients without a chest X-ray. When the tumor grows larger and significant symptoms appear, patients are usually diagnosed with Stage III or Stage IV lung cancer. Therefore, among common cancers, lung cancer has the lowest overall five-year survival rate. If patients with terminal lung cancer can be effectively diagnosed at an early stage, it will greatly help improve the overall survival rates.

The known therapies for lung cancer include surgery, radiotherapy, chemotherapy, targeted therapy and immunotherapy, wherein chemotherapy is the primary therapy for NSCLC patients. The study found that about 10% to 30% of lung cancer patients have an EGFR gene mutation, and in Asian populations, the mutation rate is even reaching 30% to 40%. The EGFR gene mutation leads to an overactivation of the EGFR signaling pathway, thereby promoting the growth of lung cancer cells. As a result, lung cancer patients with an EGFR gene mutation often have a poorer prognosis and lower survival rate than those without the gene mutation.

Regarding targeted therapy for lung cancer, it is known that EGFR-TKIs (e.g., gefitinib and erlotinib) can effectively block the EGFR signaling pathway. However, further studies have shown that after a period of targeted therapy, most patients develop acquired resistance to the drugs, wherein about 50% to 60% of NSCLC patients with a resistance to EGFR-TKIs harbor the T790M mutation in the EGFR gene.

Given the aforementioned challenges in lung cancer treatments, it would be a great news to lung cancer patients if a medicament or method being efficient in the treatment of a lung cancer which has an EGFR gene mutation or is resistant to EGFR-TKIs can be developed.

### SUMMARY OF THE INVENTION

Inventors of the present invention found that amino-chelated zinc has an excellent effect on inhibiting the growth of lung cancer cells (particularly lung cancer cells with an EGFR gene mutation), inhibiting the expression level of E-cadherin (ECAD), and inhibiting the phosphorylation of protein kinase B (AKT) and EGFR protein, thereby reducing adhesion ability of lung cancer cells, promoting lung cancer cell apoptosis, and delaying malignant progression of lung cancer. Accordingly, amino-chelated zinc can be used for treating a lung cancer.

Therefore, an objective of the present invention is to provide use of an amino acid-chelated zinc in the manufacture of a pharmaceutical composition, wherein the composition is used for treating a lung cancer. In some embodiments of the present invention, the amino acid-chelated zinc has a molar ratio of zinc to amino acid ranging from 1:1 to 1:10. In some embodiments of the present invention, the amino acid-chelated zinc is selected from the group consisting of glycine-chelated zinc, alanine-chelated zinc, valine-chelated zinc, leucine-chelated zinc, isoleucine-chelated zinc and combinations thereof.

Another objective of the present invention is to provide a pharmaceutical composition for treating a lung cancer, comprising the amino acid-chelated zinc as mentioned above. In some embodiments of the present invention, the pharmaceutical composition comprises the amino acid-chelated zinc as mentioned above and a pharmaceutically acceptable excipient, or the pharmaceutical composition consists of the amino acid-chelated zinc as mentioned above and a pharmaceutically acceptable excipient.

In some embodiments of the use and pharmaceutical composition in accordance with the present invention, the pharmaceutical composition is used for delaying malignant progression of lung cancer.

In some embodiments of the use and pharmaceutical composition in accordance with the present invention, the pharmaceutical composition is used for at least one of inhibiting lung cancer cell growth, reducing adhesion ability of lung cancer cells, and promoting lung cancer cell apoptosis.

In some embodiments of the use and pharmaceutical composition in accordance with the present invention, the pharmaceutical composition is administered by one or more of the following administration routes: oral administration, injection, and inhalation.

Still another objective of the present invention is to provide a method for treating a lung cancer, comprising administering to a subject in need thereof an effective amount of the amino acid-chelated zinc as mentioned above. In some embodiments of the present invention, the method is for delaying malignant progression of lung cancer. In some embodiments of the present invention, the method is for at least one of inhibiting lung cancer cell growth, reducing adhesion ability of lung cancer cells, and promoting lung cancer cell apoptosis.

In some embodiments of the method in accordance with the present invention, the amino acid-chelated zinc is administered by one or more of the following administration routes: oral administration, injection, and inhalation.

In some embodiments of the use, pharmaceutical composition and method in accordance with the present invention, the lung cancer is a lung cancer with an Epidermal Growth Factor Receptor (EGFR) gene mutation. In some embodiments of the present invention, the EGFR gene mutation is L858R mutation and/or T790M mutation.

In some embodiments of the use, pharmaceutical composition and method in accordance with the present invention, the lung cancer has a drug resistance to epidermal growth factor receptor tyrosine kinase inhibitor (EGFR-TKI).

To render the above objectives, technical features and advantages of the present invention more apparent, the present invention will be described in detail with reference to some embodiments hereinafter.

### BRIEF DESCRIPTION OF THE DRAWINGS

FIG. 1 shows the microscopic images of A549 cells (i.e., human lung cancer cell line) treated with amino acid-chelated zinc at a concentration of 0 micromolar (μM), 50 μM, 100 μM, or 250 μM for 48 hours (magnification: 100×).

FIG. 2 shows the microscopic images of H1975 cells (i.e., human lung cancer cell line with EGFR gene mutations) treated with amino acid-chelated zinc at a concentration of 0 μM, 50 μM, 100 μM, or 250 μM for 48 hours (magnification: 100×).

FIGS. 3 and 4 show the cell growth ratios in A549 and H1975 cells treated with amino acid-chelated zinc at a concentration of 0 μM, 50 μM, 100 μM, 250 μM, or 500 μM for 24 hours, respectively. The cell growth ratios were determined using Cell Counting Kit-8 (CCK8), wherein based on the result of untreated cells (0 μM) (i.e., the cell growth ratio of the untreated cells was set to 1), the cell growth ratios of cells treated with different concentrations of amino acid-chelated zinc were calculated.

FIGS. 5 to 8 show the expression levels of ECAD protein, AKT protein, and phosphorylated AKT (p-AKT) protein in H1975 cells treated with amino acid-chelated zinc at a concentration of 0 μM, 50 μM, 100 μM, 250 μM, or 500 μM, wherein FIG. 5 shows the experimental results from Western blotting using GAPDH protein as an internal control; and FIGS. 6 to 8 show the relative expression levels of ECAD protein, AKT protein and p-AKT protein based on the result of untreated cells (0 μM) (i.e., the expression level of untreated cells was set to 100%), which were quantified from the experimental results shown in FIG. 5 using an image analysis software.

FIGS. 9 and 10 show the expression levels of EGFR protein and phosphorylated EGFR (p-EGFR) protein in H1975 cells untreated or treated with zinc sulfate or amino acid-chelated zinc at a concentration of 0.1 mM or 0.5 mM for 6 hours, wherein FIG. 9 shows the experimental results from Western blotting using EGFR protein as an internal control; and FIG. 10 shows the relative expression level of p-EGFR based on the result of control group (not treated with zinc sulfate or amino acid-chelated zinc) (i.e., the expression level of control group was set to 100%), which was quantified from the experimental results shown in FIG. 9 using an image analysis software.

FIGS. 11 and 12 show the expression levels of EGFR protein and p-EGFR protein in H1975 cells treated with different concentrations of zinc sulfate or amino acid-chelated zinc for 24 hours, wherein FIG. 11 shows the experimental results from Western blotting using EGFR protein as an internal control; and FIG. 12 shows the relative expression of p-EGFR based on the result of control group (not treated with zinc sulfate or amino acid-chelated zinc) (i.e., the expression level of control group was set to 100%), which was quantified from the experimental results shown in FIG. 11 using an image analysis software.

### DESCRIPTION OF THE EMBODIMENTS

Hereinafter, some embodiments of the present invention will be described in detail. However, the present invention may be embodied in various embodiments and should not be limited to the embodiments described in the specification.

Unless it is additionally explained, the expressions “a,” “the,” or the like recited in the specification (especially in the claims) should include both the singular and the plural forms. The term “treat,” “treating” or “treatment” recited herein should not be construed as referring to treat a subject until the subject has completely recovered, but should include maintaining the progression or symptoms of diseases in a substantially static state, increasing the recovery rate of a subject, alleviating the severity of a particular condition (for example, reducing the tumor size or cancer cell spread), or increasing the quality of life of a patient. The term “subject” refers to a human or a non-human mammal. Examples of the non-human mammal include, but are not limited to, bovine, horse, sheep, goat, pig, donkey, mule, dog, cat, rabbit, rodent (such as hamster, guinea pig), ape, monkey, and orangutan.

The numerical ranges (e.g., 5 to 100) used herein should be construed as including all of the rational numbers within the ranges and ranges consisting of any rational numbers within the ranges. Therefore, the numerical ranges used herein should include all the possible combinations of numerical values between the listed minimum and maximum values.

As mentioned above, inventors of the present invention found that amino-chelated zinc has excellent effects on inhibiting the growth of lung cancer cells (particularly lung cancer cells with an EGFR gene mutation), reducing adhesion ability of lung cancer cells, and promoting lung cancer cell apoptosis. Therefore, the present invention relates to use of amino acid-chelated zinc in lung cancer (particularly a lung cancer with an EGFR gene mutation or resistance to EGFR-TKIs), including: use of an amino acid-chelated zinc in the manufacture of a pharmaceutical composition for treating a lung cancer; a pharmaceutical composition for treating a lung cancer, comprising an amino acid-chelated zinc; and a method for treating a lung cancer, comprising administering to a subject in need thereof an effective amount of amino acid-chelated zinc.

### Amino Acid-Chelated Zinc

The amino acid-chelated zinc used in the present invention can be a commercially available product or a product of a synthesis method. The synthetic methods for amino acid-chelated zinc are well known in the art. For example, the chelation of amino acid and zinc can be performed by operations such as physically mixing and/or heating to provide the amino acid-chelated zinc. The amino acid-chelated zinc has a molar ratio of zinc to amino acid ranging from 1:1 to 1:10, such as 1:1, 1:2, 1:3, 1:4, 1:5, 1:6, 1:7, 1:8, 1:9 or 1:10, or within a range between any two of the values described herein. In some embodiments of the present invention, the amino acid-chelated zinc has a molar ratio of zinc to amino acid ranging from 1:1 to 1:4.

There is no specific limitation on the amino acid used in the preparation of the amino acid-chelated zinc for the present invention as long as the desired effect(s) of the present invention (i.e. treating a lung cancer) is not affected. Examples of suitable amino acid for the preparation of the amino acid-chelated zinc include, but are not limited to, glycine, alanine, valine, leucine, isoleucine and combinations thereof. The amino acid-chelated zinc used in the present invention can be prepared from a zinc-containing inorganic compound, which serves as a zinc source. Examples of the zinc-containing inorganic compound include, but are not limited to, zinc sulfate, zinc carbonate, zinc oxide, zinc chloride and combinations thereof.

In some embodiments, the amino acid-chelated zinc used in the present invention can be prepared by sequentially conducting the steps of mixing an amino acid and a zinc source, refrigerating and heating. Preferably, the refrigerating step comprises placing the mixture obtained from the mixing step at 0 °C to 4 °C for at least 8 hours, such as placing at 0 °C to 4 °C for 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, or 48 hours, or within a range between any two of the values described herein. Preferably, the heating step comprises heating the refrigerated mixture at 50 °C to 100 °C for 6 hours to 24 hours, such as heating the refrigerated mixture at 50 °C, 52.5 °C, 55 °C, 57.5 °C, 60 °C, 62.5 °C, 65 °C, 67.5 °C, 70 °C, 72.5 °C, 75 °C, 77.5 °C, 80 °C, 82.5 °C, 85 °C, 87.5 °C, 90 °C, 92.5 °C, 95 °C, 97.5 °C or 100 °C for 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours or 24 hours. Alternatively, the heating temperature and heating time can be within a range between any two of the values described herein. In some embodiments of the present invention, a drying step (e.g., thermal drying or lyophilization) can be further conducted after the heating step to provide the amino acid-chelated zinc in powder form.

### Pharmaceutical Composition for Treating a Lung Cancer

As mentioned above, inventors of the present invention found that amino-chelated zinc has a significant inhibitory effect on lung cancer cell growth, reducing adhesion ability of lung cancer cells, and promoting lung cancer cell apoptosis, and thus, amino acid-chelated zinc can be used to provide a pharmaceutical composition for treating a lung cancer. Therefore, in an aspect of the present invention, a pharmaceutical composition for treating a lung cancer is provided, wherein the pharmaceutical composition comprises an effective amount of the amino acid-chelated zinc as mentioned above.

The pharmaceutical composition provided in accordance with the present invention can be administered systemically or topically and can be delivered by various drug delivery systems (DDSs). Suitable drug delivery systems include, but are not limited to, an oral drug delivery system, an injectable drug delivery system and an inhalation drug delivery system. For example, the pharmaceutical composition can be administered to a subject in need by one or more of the following administration routes: oral administration, intravenous injection (including drip infusion and bolus injection), intramuscular injection, subcutaneous injection, arterial injection, intraperitoneal injection, nasal inhalation (e.g., nasal spray) or oral inhalation (e.g., dry powder inhaler), but is not limited thereby. In addition, to enhance bioavailability, control drug release speed, target the lesion precisely and reduce side effects, the pharmaceutical composition provided in accordance with the present invention can be delivered by liposomes, microcapsules, nanoparticles, and the like.

The pharmaceutical composition provided in accordance with the present invention can be in any suitable form without special limitation, and can be presented in an appropriate dosage form depending on the desired purpose(s). For example, the pharmaceutical composition can be available as a suspension, an emulsion, a solution (such as syrup, elixirs, tincture, etc.), coated tablets, tablets, granules, powders, capsules, pellets, pills and an injection, but is not limited thereby. Depending on the form and purpose(s), a pharmaceutically acceptable excipient can be chosen to provide the pharmaceutical composition. Suitable excipients are well known by a person skilled in the art of pharmaceutical manufacturing and include, but are not limited to, a diluent, a surfactant, a flow agent, a disintegrating agent, an adhesive, a buffer, a colorant, a flavoring agent, an antioxidant, a preservative and a film forming agent.

### Method for Treating a Lung Cancer

In another aspect of the present invention, a method for treating a lung cancer is provided, wherein the method comprises administering to a subject in need an effective amount of an amino acid-chelated zinc described above.

In the method in accordance with the present invention, the subject in need refers to a subject suffering from a lung cancer, and particularly a subject suffering from a lung cancer with an EGFR gene mutation (e.g., L858R mutation and/or T790M mutation) or resistance to EGFR-TKIs.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Preparation Example: Preparation of Amino Acid-Chelated Zinc

The amino acid-chelated zinc used in the examples is prepared by the following steps: sieving food grade zinc sulfate and food grade glycine with a 30 mesh sieve, respectively, and mixing them at a molar ratio ranging from 1:1 to 1:4; refrigerating the mixture thus obtained at 0 °C to 4 °C for 12 hours to 24 hours; and heating the refrigerated mixture at a temperature of 80 °C for 12 hours to obtain a glycine-chelated zinc powder.

### Example 1: Effect of Amino Acid-Chelated Zinc on Lung Cancer Cell Growth

### (1-1) Cell Treatment

### (1-1-1) Human Lung Cancer Cell Line, A549 Cell

A549 cells (purchased from Bioresource Collection and Research Center (BCRC), website: www.bcrc.firdi.org.tw/; no EGFR gene mutation) were seeded to a 96-well plate at a density of 5×10³ cells/well, cultured with DMEM medium supplemented with 10% fetal bovine serum (FBS; purchased from Hyclone) and 1% antibiotics (comprising 100 U/mL of penicillin, 100 μg/mL of streptomycin and 0.25 μg/mL amphotericin B; all purchased from Simply). The cells were incubated overnight in an incubator at 37 °C and 5% CO₂. After incubation, the medium in each well of the 96-well plate was replaced with DMEM medium containing 0 μM, 50 μM, 100 μM, 250 μM, or 500 μM of amino acid-chelated zinc obtained from Preparation Example. The cells were further incubated for 48 hours to obtain A549 cells treated with amino acid-chelated zinc.

### (1-1-2) Human Lung Cancer Cell Line with EGFR Gene Mutations, H1975 Cell

The cell treatment of (1-1-1) was repeated, but A549 cells were replaced with H1975 cells (purchased form American Type Culture Collection (ATCC); having L858R and T790 double mutation in EGFR gene), and the cells were cultured with RPMI-1640 medium (purchased from Hyclone) supplemented with 10% FBS and 1% antibiotics for 24 hours.

### (1-2) Appearance Observation

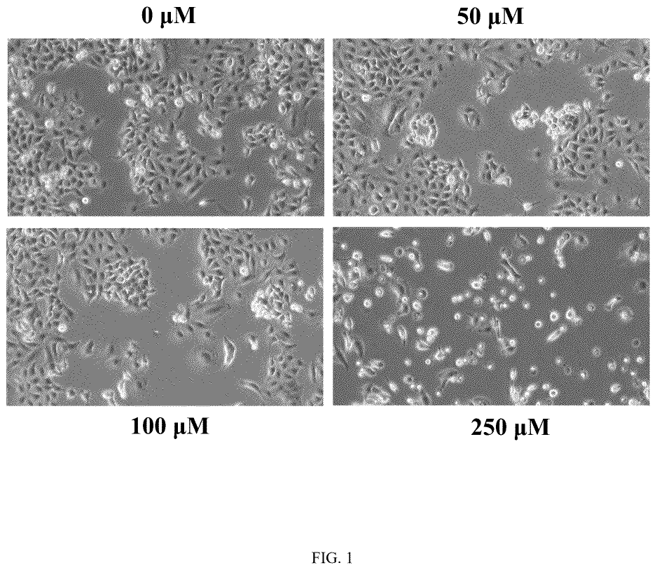

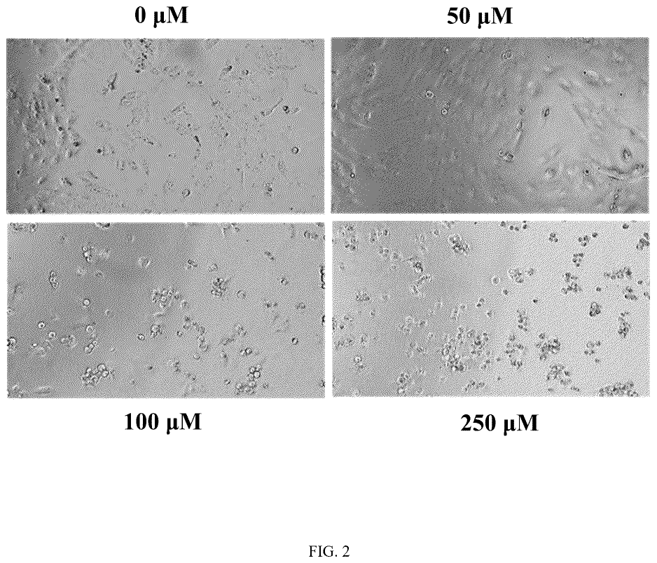

The cells obtained from (1-1) being treated with amino acid-chelated zinc at different concentrations were observed with an inverted microscope. The results are shown in FIGS. 1 and 2. FIG. 1 shows pictures of A549 cells treated with amino acid-chelated zinc at a concentration of 0 μM, 50 μM, 100 μM, or 250 μM for 48 hours, wherein the density of A549 cells treated with amino acid-chelated zinc at a concentration of 250 μM was significantly decreased compared to that of untreated cells (amino acid-chelated zinc=0 μM). FIG. 2 shows pictures of H1975 cells treated with amino acid-chelated zinc at a concentration of 0 μM, 50 μM, 100 μM, or 250 μM for 24 hours, wherein the density of H1975 cells treated with amino acid-chelated zinc at a concentration of 250 μM was also significantly decreased compared to that of untreated cells.

### (1-3) CCK-8 Assay

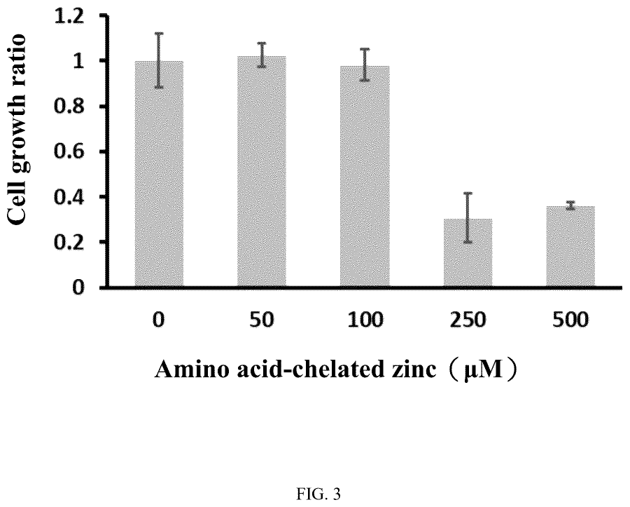

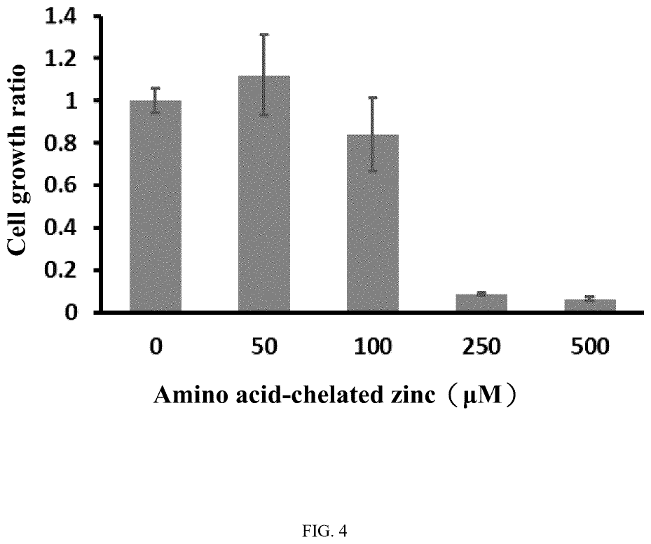

10 μL of CCK-8 reagent (purchased from Sigma) was added to each well of the 96-well plate obtained from (1-1), and the 96-well plate was placed in an incubator at 37 °C for 2 hours to 4 hours. The 96-well plate was then removed from the incubator, and the absorbance value at 450 nm (OD₄₅₀) in each well was detected using a spectrophotometer. The cell growth ratios of A549 cells (or H1975 cells) treated with amino acid-chelated zinc were calculated based on the result of untreated cells (i.e., the cell growth ratio of untreated cells was set to 1). The results are shown in FIGS. 3 and 4.

FIG. 3 shows the cell growth ratios of A549 cells treated with amino acid-chelated zinc at a concentration of 0 μM, 50 μM, 100 μM, 250 μM, or 500 μM (relative to untreated cells). As shown in FIG. 3, the growths of A549 cells treated with amino acid-chelated zinc at a concentration of 250 μM or 500 μM for 48 hours were significantly inhibited, and the inhibitory rates were up to 70%. FIG. 4 shows the cell growth ratios of H1975 cells treated with amino acid-chelated zinc at a concentration of 0 μM, 50 μM, 100 μM, 250 μM, or 500 μM, wherein the growths of H1975 cells treated with amino acid-chelated zinc at a concentration of 250 μM or 500 μM were significantly inhibited compared to that of untreated cells, and the inhibitory rates were up to 90%.

### (1-4) Conclusion

It was noted from the above experimental results that amino acid-chelated zinc has a significant inhibitory effect on lung cancer cell growth, particularly, on the growth of lung cancer cells with an EGFR gene mutation (the 24-hour treatment was sufficient to achieve a 90% inhibitory rate). These results suggest that amino acid-chelated zinc can effectively treat a lung cancer.

### Example 2: Effect of Amino Acid-Chelated Zinc on Adhesion Ability of Lung Cancer Cells and Lung Cancer Cell Apoptosis

Studies have found that ECAD and AKT serve as downstream proteins in the EGFR signaling pathway. The expression of ECAD in cells is associated with their adhesion ability, while the phosphorylation of AKT in cells is linked to cell apoptosis. In this example, the effects of amino acid-chelated zinc on adhesion ability and lung cancer cell apoptosis were evaluated by observing the expression level of ECAD and the phosphorylation of AKT in lung cancer cells treated with amino acid-chelated zinc.

### (2-1) Preparation of Protein Samples

H1975 cells were seeded into 6-cm cell culture dishes, and then cultured with the RPMI-1640 medium containing the amino acid-chelated zinc obtained from Preparation Example at a concentration of 0 μM, 50 μM, 100 μM, 250 μM, or 500 μM, respectively. After 24-hour culture, the media were removed from the dishes, and the cells in each dish were washed by phosphate buffered saline (PBS). Thereafter, 150 μL of PRO-PREP protein extraction solution (purchased from iNtRON Biotechnology) was added into each dish, and the bottom of each dish was gently scraped with a cell scraper to suspend the cells in the extraction solution. The extraction solutions containing the cells were collected from each dish and placed into new microcentrifuge tubes. The microcentrifuge tubes were centrifuged at 14,000 rpm for 10 minutes at 4 °C. Then, the supernatants obtained from the centrifugation were moved to new microcentrifuge tubes, respectively. The supernatants were subjected to protein quantification, followed by thorough mixing 10 μg of protein with protein sample buffer (F1 5×Protein Sample Buffer, purchased from FUTURE SCIENTIFIC CO., LTD). The mixtures were then heated at 100 °C for 10 minutes to obtain the protein samples of this example used in subsequent experiments.

### (2-2) Western Blotting

Anti-ECAD antibody (product No.: #3195, purchased from Cell Signaling Technology), anti-AKT antibody (product No.: #4685, purchased from Cell Signaling Technology), anti-p-AKT antibody (product No.: S473, #40660, purchased from Cell Signaling Technology), and anti-GAPDH antibody (product No.: GTX100118, purchased from GeneTex International Corporation) were used as primary antibodies, and anti-rabbit or anti-mouse antibody (purchased from Cell Signaling Technology) was used as a secondary antibody. The protein samples obtained from (2-1) were subjected to western blotting. The results are shown in FIGS. 5 to 8. GAPDH protein was used as an internal control.

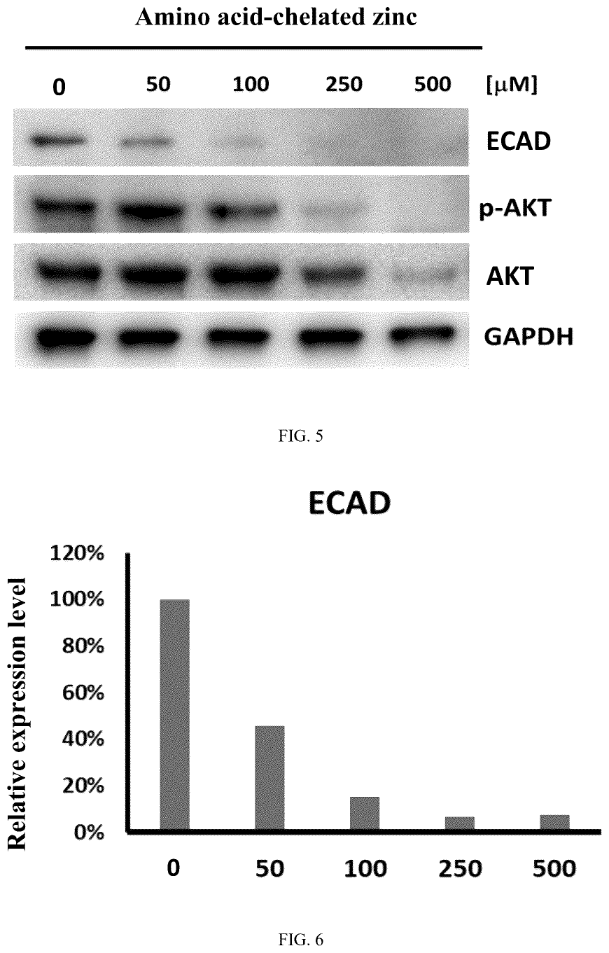

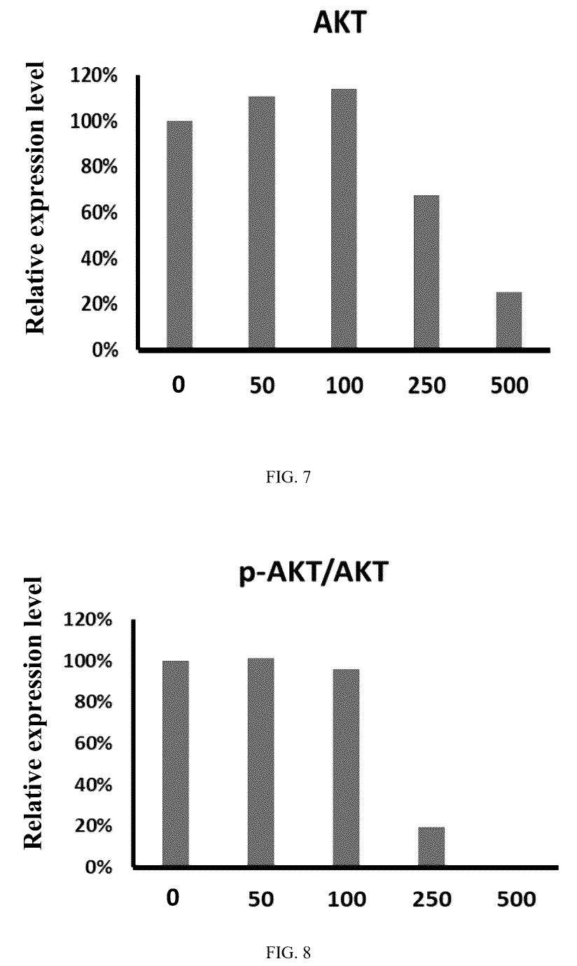

FIG. 5 shows the experimental results of western blotting, while FIGS. 6 to 8 show the relative expression levels quantified by image analysis software. As shown in FIG. 5, the expression levels of ECAD protein, AKT protein, and p-AKT protein all decreased with increasing concentrations of amino acid-chelated zinc. FIG. 6 shows that the effect of inhibiting about 60% the expression level of ECAD protein can be achieved at 50 μM of amino acid-chelated zinc, and even up to 90% at 100 μM of amino acid-chelated zinc. FIG. 8 shows that the effect of inhibiting about 80% the phosphorylation of AKT protein can be achieved at 250 μM of amino acid-chelated zinc. Although FIG. 5 and FIG. 7 show that the expression level of AKT protein decreased with increasing concentrations of amino acid-chelated zinc, FIG. 8 demonstrates that the relative expression level of p-AKT (normalized to the total expression of AKT protein) also decreased. This indicates that amino acid-chelated zinc not only inhibits the expression of the AKT protein but also suppresses its activation, thereby blocking the signal transduction involved in growth promotion and inducing apoptosis.

### (2-3) Conclusion

As demonstrated by the experimental results above, amino acid-chelated zinc is effective in inhibiting the expression levels of ECAD and AKT proteins, while inhibiting the activation of AKT protein and thus blocking the signal transduction, thereby achieving the effects on reducing adhesion ability of lung cancer cells and promoting lung cancer cell apoptosis. Accordingly, amino acid-chelated zinc is effective in treating a lung cancer, and especially a lung cancer with an EGFR gene mutation or resistance to EGFR-TKIs.

### Example 3: Effect of Amino Acid-Chelated Zinc on Malignant Progression of Lung Cancer

Studies indicated that the phosphorylation of EGFR protein is associated with the malignant progression of lung cancer. In a zinc-deficient environment, the phosphorylation of the EGFR protein increases; however, the level of EGFR phosphorylation in lung cancer cells can be restored to that observed under normal zinc conditions by supplementing the cell medium with additional zinc. In this example, the expression level of phosphorylated EGFR (p-EGFR) in lung cancer cells treated with amino acid-chelated zinc was observed to evaluate the effects of amino acid-chelated zinc on delaying the malignant progression of lung cancer.

### (3-1) Preparation of Protein Samples

H1975 cells were treated using a method similar to that of (2-1), but the cells were cultured with RPMI-1640 medium containing zinc sulfate or amino acid-chelated zinc obtained from Preparation Example at a concentration of 0.1 millimolar (mM) or 0.5 mM for 6 hours or 24 hours.

### (3-2) Western Blotting

Anti-EGFR antibody (product no. #4267, purchased from Cell Signaling Technology) and anti p-EGFR antibody (product no. Tyr1068, #48576, purchased from Cell Signaling Technology) were used as primary antibodies, and anti-rabbit or anti-mouse antibody (purchased from Cell Signaling Technology) was used as a secondary antibody. The protein samples obtained from (3-1) were subjected to western blotting. The results are shown in FIGS. 9 to 12. The total expression level of EGFR was used as an internal control.

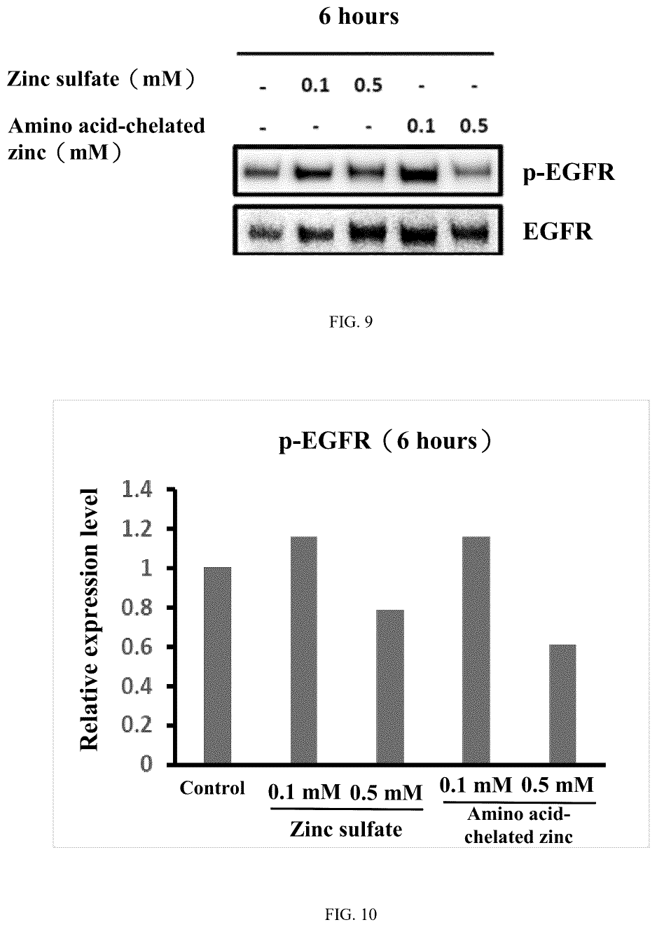

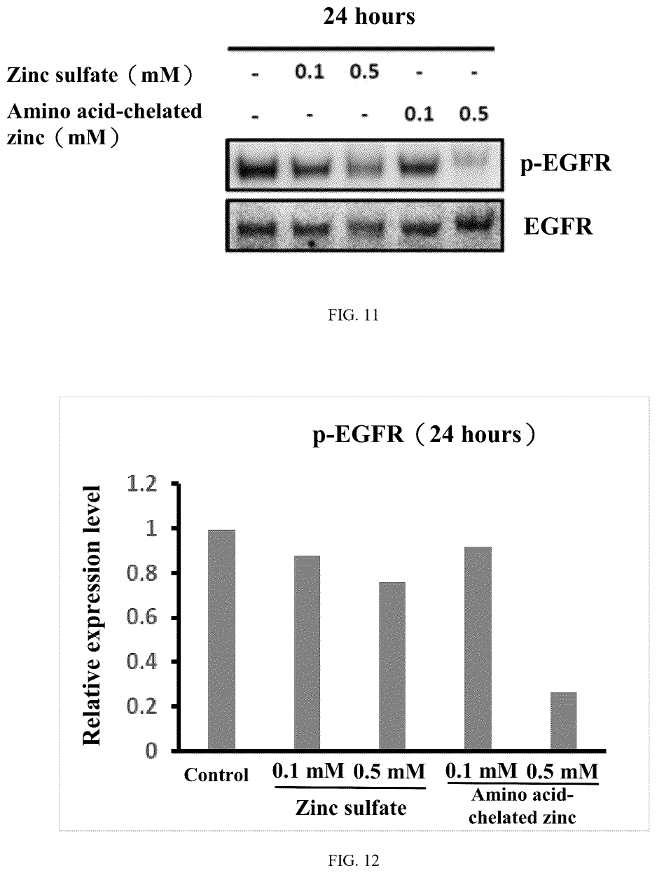

FIGS. 9 and 11 show the experimental results of Western-blotting results for cells treated for 6 hours or 24 hours, respectively. FIGS. 10 and 12 show the relative expression levels quantified from the experimental results of FIGS. 9 and 11 using image analysis software. As shown in FIGS. 9 and 10, as compared to the untreated cells (control group), both the uses of zinc sulfate and amino acid-chelated zinc at 0.5 mM markedly inhibited the phosphorylation of the EGFR protein after the 6-hour treatment, wherein zinc sulfate can achieve about 25% inhibitory rate and amino acid-chelated zinc can achieve about 40% inhibitory rate. FIGS. 11 and 12 show the results from the cells treated for 24 hours. As shown by FIGS. 11 and 12, as compared to control group (untreated cells), the use of 0.5 mM zinc sulfate can inhibit about 25% of the phosphorylation of the EGFR protein and the use of 0.5 mM of amino acid-chelated zinc can inhibit about 80% of the phosphorylation of the EGFR.

### (3-3) Conclusion

As demonstrated by the above experimental results, zinc sulfate can inhibit about 25% EGFR phosphorylation after both 6-hour and 24-hour treatments; however, the inhibitory effect of amino acid-chelated zinc increased with treatment time, rising from about 40% to 80%. This indicates that amino acid-chelated zinc has a superior effect on inhibiting the phosphorylation of EGFR protein (better than that of zinc sulfate), thereby delaying the malignant progression of lung cancer. Accordingly, amino acid-chelated zinc is effective in treating a lung cancer.

Based on the experimental results illustrated by the examples, amino acid-chelated zinc is not only effective in inhibiting lung cancer cell growth, especially effective in inhibiting the growth of lung cancer cells with an EGFR gene mutation, but also effective in decreasing the expression level of the ECAD protein, and is able to decrease the phosphorylation of the AKT protein and EGFR protein, thereby reducing adhesion ability of lung cancer cells, promoting lung cancer cell apoptosis and delaying the malignant progression of lung cancer. Therefore, amino acid-chelated zinc can be used in treating a lung cancer and particularly a lung cancer with an EGFR gene mutation or resistance to EGFR-TKIs.

What is claimed is:

1. A method for treating a lung cancer, comprising administering to a subject in need thereof an effective amount of an amino acid-chelated zinc.

2. The method of claim 1, wherein the amino acid-chelated zinc has a molar ratio of zinc to amino acid ranging from 1:1 to 1:10.

3. The method of claim 1, wherein the amino acid-chelated zinc is selected from the group consisting of glycine-chelated zinc, alanine-chelated zinc, valine-chelated zinc, leucine-chelated zinc, isoleucine-chelated zinc and combinations thereof.

4. The method of claim 1, wherein the lung cancer is a lung cancer with an Epidermal Growth Factor Receptor (EGFR) gene mutation.

5. The method of claim 4, wherein the EGFR gene mutation is L858R mutation and/or T790M mutation.

6. The method of claim 1, wherein the lung cancer has a drug resistance to epidermal growth factor receptor tyrosine kinase inhibitor (EGFR-TKI).

7. The method of claim 1, which is for delaying malignant progression.

8. The method of claim 2, which is for delaying malignant progression.

9. The method of claim 3, which is for delaying malignant progression.

10. The method of claim 4, which is for delaying malignant progression.

11. The method of claim 6, which is for delaying malignant progression.

12. The method of claim 1, which is for at least one of inhibiting lung cancer cell growth, reducing adhesion ability of lung cancer cells, and promoting lung cancer cell apoptosis.

13. The method of claim 2, which is for at least one of inhibiting lung cancer cell growth, reducing adhesion ability of lung cancer cells, and promoting lung cancer cell apoptosis.

14. The method of claim 3, which is for at least one of inhibiting lung cancer cell growth, reducing adhesion ability of lung cancer cells, and promoting lung cancer cell apoptosis.

15. The method of claim 4, which is for at least one of inhibiting lung cancer cell growth, reducing adhesion ability of lung cancer cells, and promoting lung cancer cell apoptosis.

16. The method of claim 6, which is for at least one of inhibiting lung cancer cell growth, reducing adhesion ability of lung cancer cells, and promoting lung cancer cell apoptosis.

17. The method of claim 1, the amino acid-chelated zinc is administered by one or more of the following administration routes: oral administration, injection, and inhalation.

18. The method of claim 2, the amino acid-chelated zinc is administered by one or more of the following administration routes: oral administration, injection, and inhalation.

19. The method of claim 3, the amino acid-chelated zinc is administered by one or more of the following administration routes: oral administration, injection, and inhalation.

20. The method of claim 4, the amino acid-chelated zinc is administered by one or more of the following administration routes: oral administration, injection, and inhalation.
